(19) United States
(12) Patent Application Publication
(10) Pub. No.: US 2013/0085553 A1
(43) Pub. Date: Apr. 4, 2013

(54) FUNCTIONAL ABDOMINAL BELT

(71) Applicant: Sungwon Kang, Incheon (KR)

(72) Inventor: Sungwon Kang, Incheon (KR)

(21) Appl. No.: 13/704,550

(22) PCT Filed: May 30, 2011
(86) PCT No.: PCT/KR2011/003959

§ 371 Date: Dec. 14, 2012

(30) Foreign Application Priority Data
Jun. 14, 2010 (KR) 10-2010-0056211

(51) Int. Cl. A61N 5/00; A61F 7/08

(52) U.S. Cl. CPC A61N 5/00; A61F 7/08
USPC 607/100

(57) ABSTRACT

The present invention relates to a functional health band, including a health band unit configured to have a first pocket formed at a center of a body, wherein the first pocket has an opening having a rear side thereof opened, and to have first and second fastening units provided at both ends of the body and a main body inserted into the first pocket in attachable and detachable manners and configured to comprise a low frequency generation unit for generating a low frequency and a heat generation unit for generating hot heat. If sweat is soaked in or alien substances are adhered to the health band unit after the functional health band is used for a specific duration of time, the main body can be separated from the health band unit and washed sanitarily.

### BACKGROUND OF THE INVENTION

1. Technical Field

The present invention relates to a functional health band and, more particularly, to a functional health band which is configured to perform low frequency therapies or thermotherapy selectively and is easily washed and sanitary and which facilitates manipulations for low frequency therapies or thermotherapy, checks information on the human body, such as body temperature, the pulse, and body fat, and provides supplementary functions, such as a step counter, an MP3 player, and a digital watch.

2. Description of the Related Art

In general, a health band is used to correct the bodily shape of an abdomen of the human body and used for treatment. A variety of functional health bands for low frequency therapies and thermotherapy are being released.

There was suggested Korean Utility Model Registration No. 0404206 as an example of this functional health band. The health band suggested by the registered Utility Model relates to a health band for a low frequency therapy machine capable of thermotherapy fomentation. The suggested health band includes a low frequency generator mounted on one side within the health band closely adhered to the waist of a user, a plurality of low frequency pads electrically connected to the low frequency generator, arranged on the inside of the health band, and configured to come in contact with the body of a user and output a low frequency, a hot wire arranged on the rear of the low frequency pads and configured to heat the low frequency pads at specific temperature, and a microcomputer electrically connected to the hot wire and configured to supply or block a power source in an electrical cycle, such as a low frequency pulse generated from the low frequency generator.

The health band suggested by the registered Utility Model can perform low frequency therapies and thermotherapy, but has a structural problem in that washing is impossible because the low frequency pads for outputting a low frequency, the hot wire for generating heat, and the microcomputer responsible for control are integrally provided on the inside of the health band. In particular, for the purpose of low frequency therapies, the health band has to be directly closed to the abdomen of the body. In this case, the health band has an insanitary problem because it is used by other persons.

Furthermore, the conventional health band is problematic in that it is difficult for a user to manipulate and check thermotherapy temperature because the user has to control the microcomputer directly with the health band worn on the abdomen.

### SUMMARY OF THE INVENTION

Accordingly, the present invention has been made keeping in mind the above problems occurring in the prior art, and an object of the present invention is to provide a functional health band which can be easily washed because a low frequency and thermotherapy main body for low frequency therapies and thermotherapy can be separated from the health band.

Another object of the present invention is to provide a functional health band equipped with wristwatch type remote control means which can be worn on the wrist in order to facilitate manipulation for low frequency therapies or thermotherapy.

Yet another object of the present invention is to provide a functional health band by providing the wristwatch type remote control means with a function of checking information on the human body, such as body temperature, the pulse, and body fat, and supplementary functions, such as a step counter, an MP3 player, and a digital watch so that the functional health band can be used in various manners.

In order to accomplish the above objects, the present invention provides a functional health band, including a health band unit configured to have a first pocket formed at the center of a body, wherein the first pocket has an opening having the rear side thereof opened, and to have first and second fastening units provided at both ends of the body and a main body inserted into the first pocket in attachable and detachable manners and configured to comprise a low frequency generation unit for generating a low frequency and a heat generation unit for generating hot heat.

Here, the main body includes a low frequency generation unit provided within a housing and configured to generate the low frequency, a heat generation unit configured to generate the hot heat, a first control unit configured to control the low frequency generation unit and the heat generation unit, a first power source unit configured to supply the driving power source to the first control unit, a manipulation unit configured to control the driving of the low frequency generation unit and the heat generation unit, a display unit configured to display the driving states of the low frequency generation unit and the heat generation unit, and electrode terminals provided on the rear side of the housing and configured to output a signal of the low frequency to the low frequency electrode pad.

In particular, the manipulation unit includes a low frequency generation button and a thermotherapy button for manipulating the driving of the low frequency generation unit and the heat generation unit, a control button for controlling a range of the low frequency of the low frequency generation unit and setting temperature of the heat generation unit, and a first power switch for manipulating the input of the driving power source to the first power source unit.

Furthermore, the first power source unit includes a charger embedded within the housing and a jack insertion hole configured to have the coupling jack of an adapter for converting commercial power into DC power connected thereto.

Furthermore, the main body is controlled by remote control means, including a first wireless communication unit configured to receive control signals for controlling the low frequency generation unit and the heat generation unit and a second wireless communication unit configured to transmit the control signals to the first wireless communication unit wirelessly.

Furthermore, the remote control means includes a remote controller configured to control the driving of the low frequency generation unit and the heat generation unit of the main body and a band configured to have the remote controller fixed thereto and to be worn in a wrist of a user. The remote controller includes a manipulation display unit provided in a casing and configured to manipulate the driving of the low frequency generation unit and the heat generation unit of the main body and to display a setting state, a second control unit configured to output the control signals for controlling the low frequency generation unit and the heat generation unit by way of a manipulation of the manipulation display unit, the second wireless communication unit configured to wirelessly transmit the control signals to the first wireless communication unit of the main body, a second power source unit configured to supply the driving power source to the second control unit, and a second power switch configured to manipulate the input of the power to the second power source unit.

Here, the manipulation display unit includes a touch screen configured to be capable of key input and to display control and setting states of the second control unit.

Furthermore, the remote controller further includes a temperature detection unit configured to measure body temperature of a user, a pulse detection unit configured to detect a number of pulses, a body fat measurement unit configured to measure body fat of a user, an MP3 driving unit configured to provide a music or language study function, a step counter sensor unit configured to count and store a number of steps when detecting information on a step of a user, a voice output unit configured to have an earphone for outputting music or language study in a voice form connected thereto, and a timer unit configured to display the time.

Furthermore, a first pocket is provided on an upper side at the center of the body of the health band unit, and a second pocket into which the main body is inserted is further provided under the first pocket.

Furthermore, a first pocket is provided on an upper side at the center of the body of the health band unit, and a second pocket into which the main body is inserted is further provided under the first pocket.

### BRIEF DESCRIPTION OF THE DRAWINGS

FIG. 1 is a diagram showing a state in which a functional health band in accordance with the present invention is worn.

FIG. 2 is an exploded perspective view of the health band unit and a main body in accordance with the present invention.

FIG. 3 is a front view showing a state in which the main body has been inserted into the health band unit in accordance with the present invention.

FIG. 4 is a cross-sectional view of one side of the health band in accordance with the present invention.

FIG. 5 is a diagram showing remote control means in accordance with the present invention.

FIG. 6 is a control block diagram of the functional health band in accordance with the present invention.

FIG. 7 is a perspective view showing another embodiment of a health band in accordance with the present invention.

FIG. 8 is a front view showing a state in which a main body has been inserted into the health band unit of FIG. 7.

FIG. 9 is a cross-sectional view of one side of the health band shown in FIG. 7.

### DESCRIPTION OF EMBODIMENTS

Hereinafter, functional health bands in accordance with the present invention are described in detail with reference to the accompanying drawings.

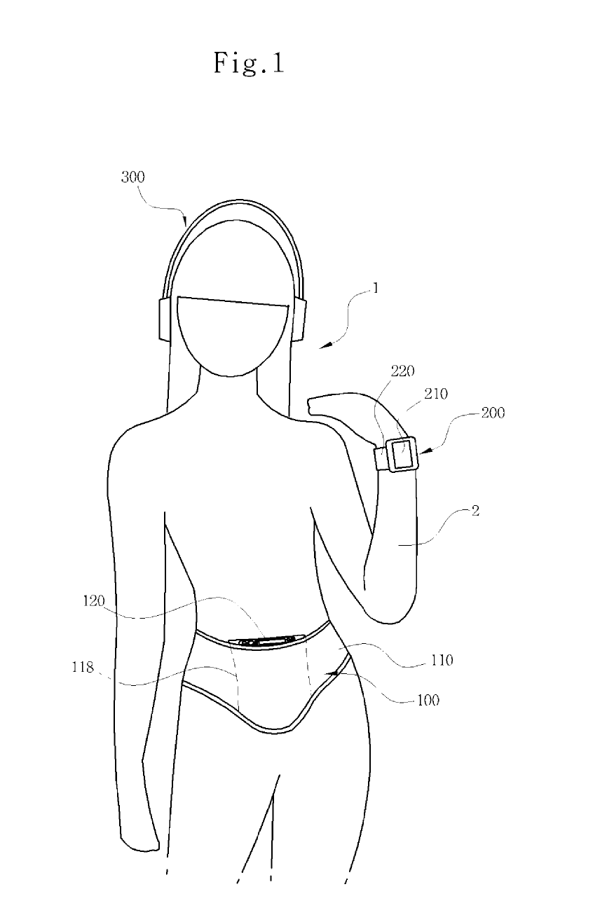

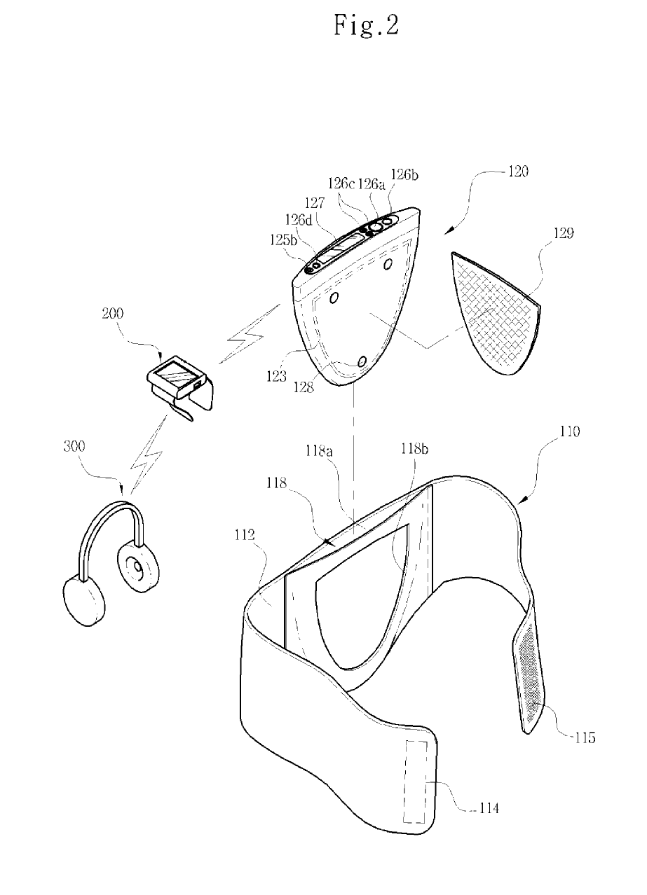

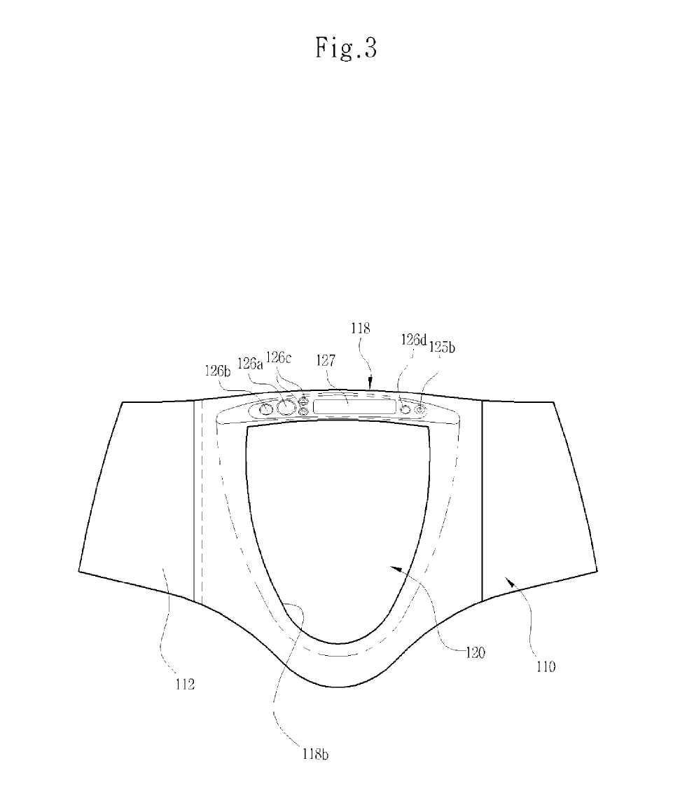

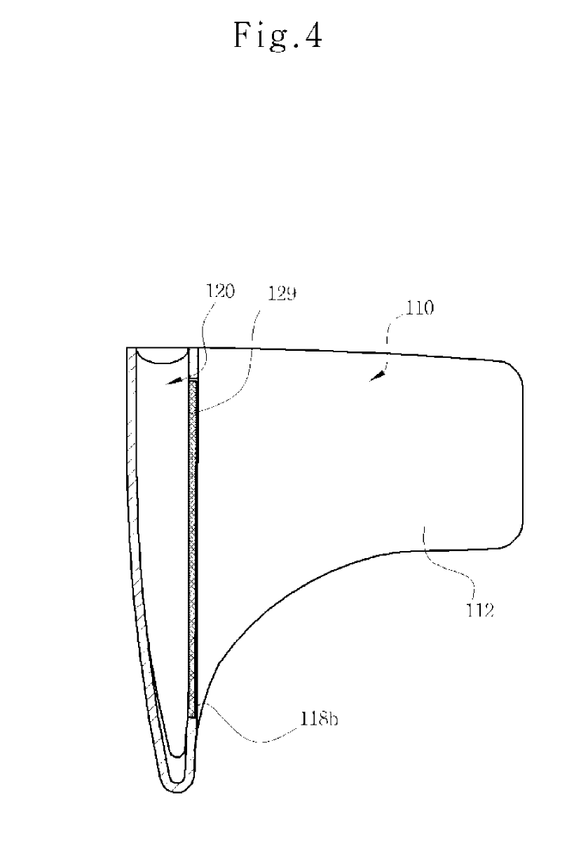

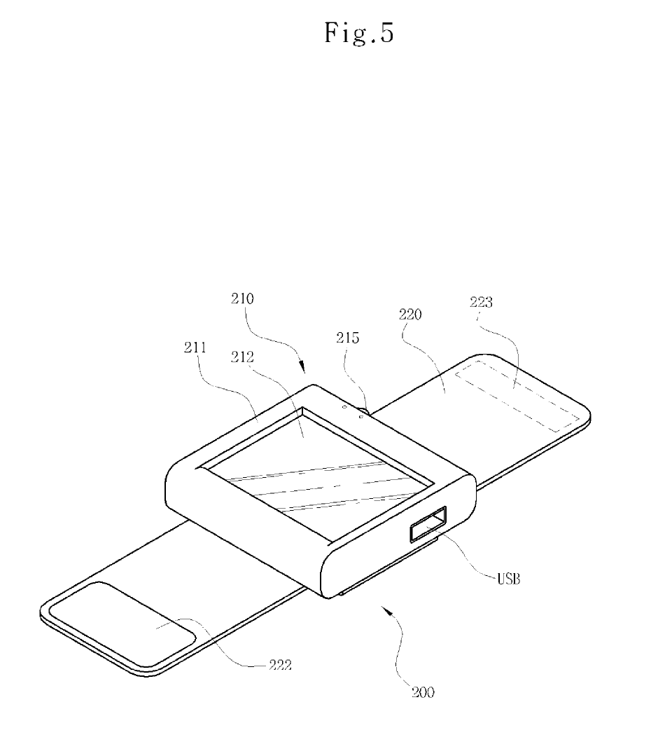

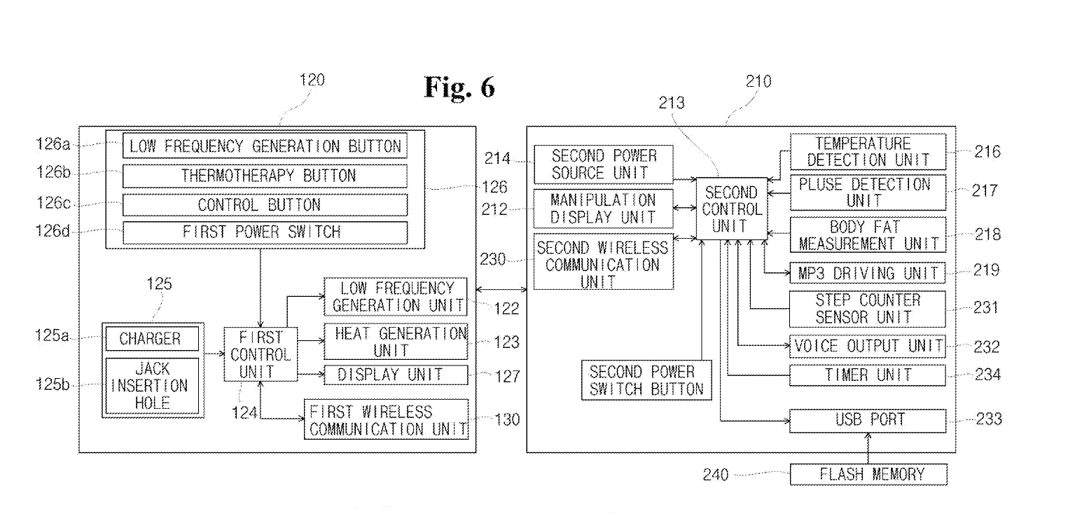

Referring to FIGS. 1 to 6, a functional health band 100 in accordance with the present invention has a low frequency or thermotherapy function and has a structure in which a main body 120 for generating a low frequency and heat can be attached to and detached from a health band unit 110.

The manipulation and control states of the low frequency and heat generation function of the main body 120 can be monitored by remote control means 200 that can be worn on the wrist 2 of a user 1 like a wristwatch.

Each of the elements of the present invention is described in detail below.

First, the health band unit 110 is made of various materials, such as cotton, synthetic fiber, or leather. First and second fastening units 114 and 115 are provided at both ends of a body 112.

The first and the second fastening units 114 and 115 can be formed of various fastening means, such as buttons or zippers, but they preferably have a velcro type so that the first and the second fastening units 114 and 115 can be easily attached and detached. It is also preferred that two or more elastic bands be provided between the first and the second fastening units 114 and 115 and the body 112 that comes in contact with the abdomen. A first pocket 118 is formed on the upper side at the center of the body 112 of the health band unit 110, and an entrance 118a into which the main body 120 can be inserted is formed in the first pocket 118. Accordingly, the main body 120 can be inserted into or separated from the first pocket 118.

Furthermore, an opening 118b is formed on the rear side of the first pocket 118. When the main body 120 is inserted into the first pocket 118, the rear part of the main body 120 can be directly adhered to the abdomen closely.

Meanwhile, the main body 120 inserted into the first pocket 118 of the health band unit 110 includes a low frequency generation unit 122 included within a housing 121 and configured to output a low frequency, a heat generation unit 123 configured to generate hot heat, a first control unit 124 configured to control the low frequency generation unit 122 and the heat generation unit 123, a first power source unit 125 configured to supply a driving power source to the first control unit 124, a manipulation unit 126 configured to control the driving of the low frequency generation unit 122 and the heat generation unit 123, and a display unit 127 configured to display the driving states of the low frequency generation unit 122 and the heat generation unit 123.

The low frequency generation unit 122 generates a low frequency of 10 to 1,000 Hz. The low frequency generation unit 122 is included within the main body 120, and electrode terminals 128 for outputting the low frequency are exposed and provided on the rear side of the housing 121.

Accordingly, when a low frequency electrode pad 129 is attached to the rear side of the housing 121 and a low frequency is applied to the abdomen, the nerves and muscle of the abdomen are stimulated, thereby being capable of performing low frequency therapies. The low frequency electrode pad 129 is a known pad in which adhesive hydrogel is coated on a surface of a conductive pads and is closely adhered to the abdomen.

Furthermore, the heat generation unit 123 includes a hot wire embedded in the rear part of the housing 121, and temperature of the heat generation unit 123 is controlled under the control of the first control unit 124. Here, temperature of the first control unit 124 is controlled by manipulating the manipulation unit 126.

Furthermore, the manipulation unit 126 includes a low frequency generation button 126a and a thermotherapy button 126b for controlling the driving of the low frequency generation unit 122 and the heat generation unit 123, a control button 126c for controlling a frequency range of the low frequency generation unit 122 and temperature of the heat generation unit 123, and a first power switch 126d for manipulating the input of power to the first power source unit 125.

Meanwhile, the display unit 127 is configured to display the driving states and frequency and temperature control states of the low frequency generation unit 122 and the heat generation unit 123 and preferably is formed of an LCD.

Furthermore, the first power source unit 125 includes a charger 125a embedded within the housing 121, an adapter (not shown) configured to convert commercial power into DC power, and a jack insertion hole 125b configured to have a coupling jack connected thereto.

In order to use the low frequency and heat generation functions, a power source supplied through the adapter can be directly used or the charged power of the charger 125a supplied through the adapter can be used.

A first wireless communication unit 130 for monitoring the control and state of the main body 120 using the remote control means 200 which can be worn in the wrist 2 is further provided in the main body 120. The remote control means 200 is equipped with a second wireless communication unit 230 for wirelessly transmitting and receiving signals to and from the first wireless communication unit 130. The first and the second wireless communication units 130 and 230 preferably are made of Bluetooth modules.

Accordingly, when the buttons of the manipulation unit 126 of the main body 120 are controlled, a control signal is wirelessly transmitted through the first wireless communication unit 130. The control signal is received through the second wireless communication unit 230 of the remote control means 200, so that the manipulation states of the low frequency generation unit 122 and the heat generation unit 123 of the main body 120 are displayed in the manipulation display unit 212 of a remote controller 210.

Meanwhile, the remote control means 200 includes the remote controller 210 configured to control the driving of the low frequency generation unit 122 and the heat generation unit 123 of the main body 120 and a band 220 configured to have the remote controller 210 fixed thereto and to be worn in the wrist of a user.

The remote controller 210 includes the manipulation display unit 212 provided in a casing 211 and configured to manipulate the driving of the low frequency generation unit 122 and the heat generation unit 123 of the main body 120 and to display a setting state, a second control unit 213 configured to output a control signal for controlling the low frequency generation unit 122 and the heat generation unit 123 by way of a manipulation of the manipulation display unit 212, the second wireless communication unit 230 configured to wirelessly transmit the control signal to the first wireless communication unit 130 of the main body 120, a second power source unit 214 configured to supply a driving power source to the second control unit 213, and a second power switch 215 configured to manipulate the input of the power to the second power source unit 214.

Furthermore, the band 220 is equipped with third and fourth fastening units 222 and 223. Like in the first and the second fastening units 114 and 115 of the health band unit 110, various fastening means, such as buttons or zippers, can be used as the third and fourth fastening units 222 and 223, but the third and fourth fastening units 222 preferably have a velcro type so that they can be easily attached and detached.

The manipulation display unit 212 is formed of a touch screen for performing a variety of key inputs and displaying the control and setting states of the second control unit 213.

Furthermore, a battery or a charger can be used as the second power source unit 214.

Meanwhile, the remote controller 210 further includes a temperature detection unit 216 configured to measure the body temperature of a user, a pulse detection unit 217 configured to detect the number of pulses, a body fat measurement unit 218 configured to measure the body fat of a user, an MP3 driving unit 219 configured to provide a music or language study function, a step counter sensor unit 231 configured to count and store the number of steps when detecting information on the step of a user, a voice output unit 232 configured to have an earphone 300 for outputting music or language study in a voice form connected thereto, and a timer unit 234 configured to display the time. In this case, the earphone 300 can be connected to the voice output unit 232 in a wired manner, but a Bluetooth module preferably is embedded in the earphone 300 so that voice information can be received wirelessly.

Accordingly, the functional health band of the present invention can be used in various manners because information on the human body, such as the body temperature, pulse, or body fat of a user, and supplementary functions, such as a step counter, an MP3 player, and a digital watch, can be displayed in the manipulation display unit 212 or outputted in a voice form through the earphone 300.

The remote controller 210 further includes a USB port 233 to which known flash memory 240 for storing MP3 files can be connected.

An example in which the functional health band in accordance with the present invention is used is described below with reference to FIGS. 1 to 6.

First, in order to apply thermotherapy to the abdomen by using the functional health band 100 in accordance with the present invention, in the state in which the main body 120 has been simply inserted into the first pocket 118 of the health band unit 110 and the first power switch 126d is turned on, the heat generation unit 123 is driven in a proper temperature range by manipulating the thermotherapy button 126b and the control button 126c.

Here, the low frequency and heat generation functions (i.e., a thermotherapy function) of the main body 120 can be manipulated conveniently in the state in which the remote control means 200 is worn in the wrist 2.

Meanwhile, in order to use the low frequency function, the low frequency electrode pad 129 is attached to the rear side of the housing 121 of the main body 120.

Even in this case, the low frequency generation unit 122 can be driven in a proper frequency range by manipulating the low frequency generation button 126a and the control button 126c provided in the main body 120. Furthermore, the low frequency function can be manipulated conveniently in the state in which the remote control means 200 is worn in the wrist 2.

While using the functional health band 100, information on the human body, such as the body temperature, pulse, or body fat of a user, and supplementary functions, such as a step counter, an MP3 player, and a watch, can be used.

Meanwhile, if sweat is soaked in or alien substances are adhered to the health band unit 110 after the functional health band 100 is used for a specific duration of time, the main body 120 can be separated from the first pocket 118 and washed sanitarily.

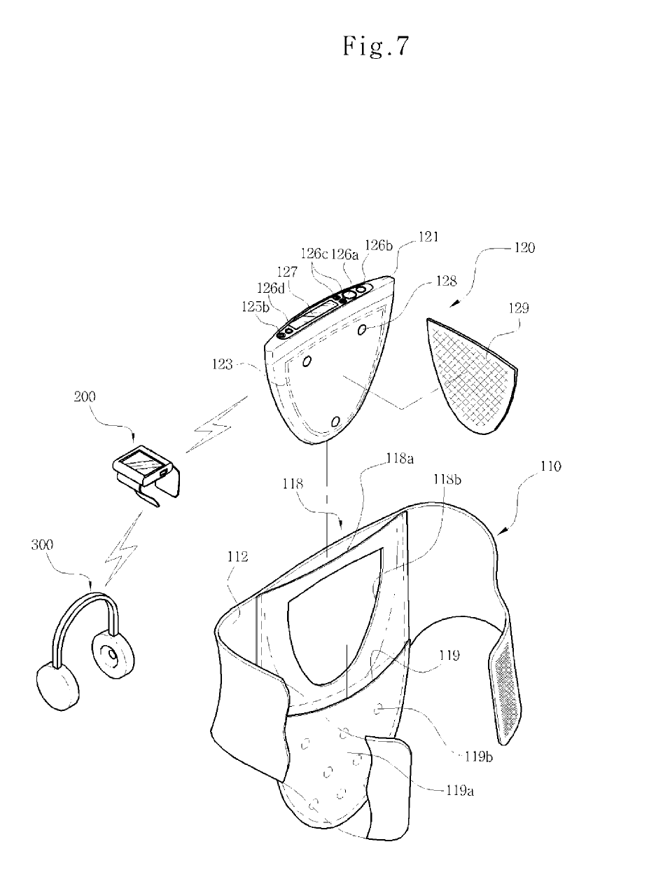

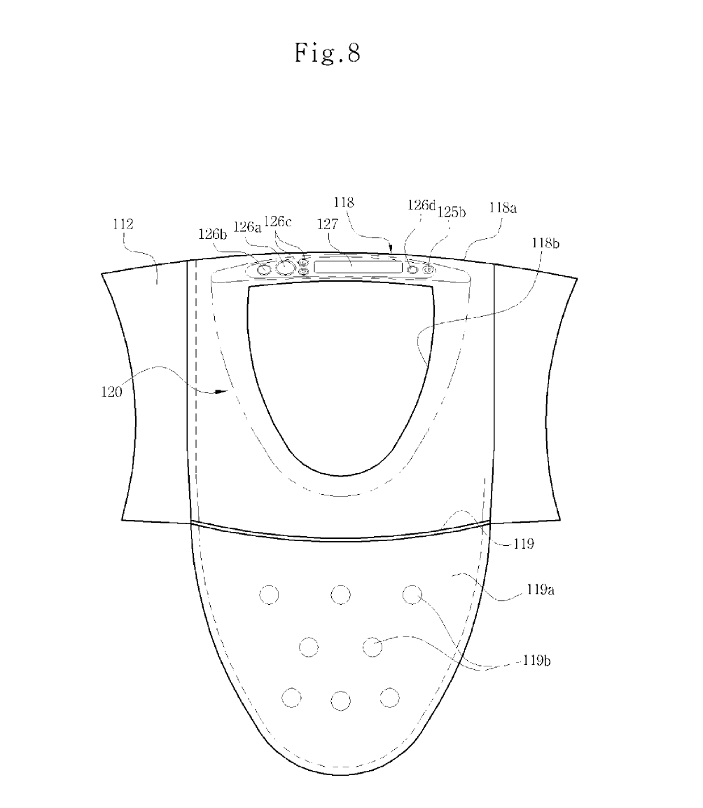

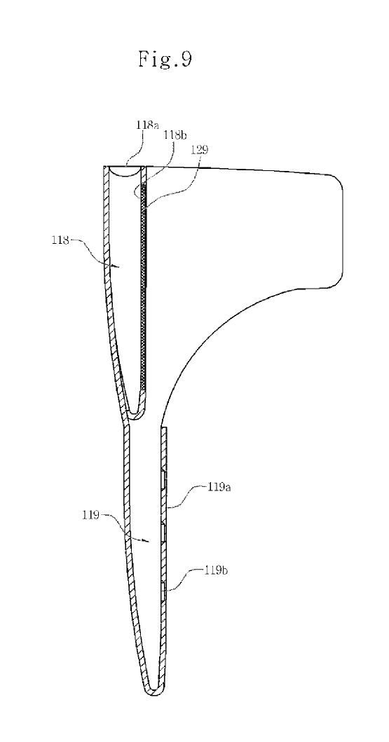

Furthermore, FIGS. 7 to 9 are diagrams illustrating a health band unit 110 in accordance with another embodiment of the present invention. Referring to FIGS. 7 to 9, the health band unit 110 includes a first pocket 118 provided on the upper side at the center of a body 112 and a second pocket 119 provided on the lower side of the first pocket 118 and configured to have the main body 120 inserted therein.

Here, an opening 118b is formed on the rear side of the first pocket 118 so that the rear part of the main body 120 can be directly adhered to the abdomen closely when the main body 120 is inserted into the first pocket 118. The rear side 119a of the second pocket 119 is closed, and permanent magnets 119b are integrally attached to the rear side 119a of the second pocket 119. The permanent magnets 119b function to help improve the circulation of the blood at the lower part of the abdomen under the navel.

Meanwhile, if the main body 120 is inserted into the first pocket 118, the thermotherapy and low frequency functions can be used selectively. If the main body 120 is inserted into the second pocket 119, however, only the thermotherapy function is used.

In accordance with the present invention, if sweat is soaked in or alien substances are adhered to the health band unit after the functional health band is used for a specific duration of time, the main body can be separated from the health band unit and washed sanitarily. Accordingly, there is an advantage in that the functional health band of the present invention is sanitary.

Furthermore, in order to control the low frequency or thermotherapy function of the main body, manipulation and check can be performed conveniently in the state in which the remote control means has been worn in the wrist. Furthermore, a function of checking information on the human body, such as body temperature, the pulse, or body fat, and supplementary functions, such as a step counter, an MP3 player, and a digital watch, are included in the remote control means. Accordingly, there is an advantage in that the functional health band of the present invention can be used in various manners.

As described above, the embodiments of the present invention have been described in detail, but the scope of the present invention is not limited to the embodiments. The scope of the present invention cover equivalents which substantially fall within the embodiments of the present invention.

What is claimed is:

1. A functional health band, comprising a health band unit 110 configured to have a first pocket 118 formed at a center of a body 112, wherein the first pocket 118 has an opening 118b having a rear side thereof opened, and to have first and second fastening units 114 and 115 provided at both ends of the body 112 and a main body 120 inserted into the first pocket 118 in attachable and detachable manners and configured to comprise a low frequency generation unit 122 for generating a low frequency and a heat generation unit 123 for generating hot heat,
wherein the main body 120 comprises a low frequency generation unit 122 provided within a housing 121 and configured to generate the low frequency, a heat generation unit 123 configured to generate the hot heat, a first control unit 124 configured to control the low frequency generation unit 122 and the heat generation unit 123, a first power source unit 125 configured to supply a driving power source to the first control unit 124, a manipulation unit 126 configured to control a driving of the low frequency generation unit 122 and the heat generation unit 123, a display unit 127 configured to display driving states of the low frequency generation unit 122 and the heat generation unit 123, and electrode terminals 128 provided on a rear side of the housing 121 and configured to output a signal of the low frequency to the low frequency electrode pad 129; and
the main body 120 is controlled by remote control means 200, comprising a first wireless communication unit 130 configured to receive control signals for controlling the low frequency generation unit 122 and the heat generation unit 123 and a second wireless communication unit 230 configured to transmit the control signals to the first wireless communication unit 130 wirelessly.

2. The functional health band of claim 1, wherein the manipulation unit 126 comprises:
a low frequency generation button 126a and a thermotherapy button 126b for manipulating the driving of the low frequency generation unit 122 and the heat generation unit 123,
a control button 126c for controlling a range of the low frequency of the low frequency generation unit 122 and setting temperature of the heat generation unit 123, and
a first power switch 126d for manipulating the input of the driving power source to the first power source unit 125.

3. The functional health band of claim 1, wherein the first power source unit 125 comprises:
a charger 125a embedded within the housing 121, and a jack insertion hole 125b configured to have a coupling jack of an adapter for converting commercial power into DC power connected thereto.

4. The functional health band of claim 1, wherein:
the remote control means 200 comprises a remote controller 210 configured to control the driving of the low frequency generation unit 122 and the heat generation unit 123 of the main body 120 and a band 220 configured to have the remote controller 210 fixed thereto and to be worn in a wrist of a user; and
the remote controller 210 comprises a manipulation display unit 212 provided in a casing 211 and configured to manipulate the driving of the low frequency generation unit 122 and the heat generation unit 123 of the main body 120 and to display a setting state, a second control unit 213 configured to output the control signals for controlling the low frequency generation unit 122 and the heat generation unit 123 by way of a manipulation of the manipulation display unit 212, the second wireless communication unit 230 configured to wirelessly transmit the control signals to the first wireless communication unit 130 of the main body 120, a second power source unit 214 configured to supply the driving power source to the second control unit 213, and a second power switch 215 configured to manipulate the input of the power to the second power source unit 214.

5. The functional health band of claim 4, wherein the manipulation display unit 212 comprises a touch screen configured to be capable of key input and to display control and setting states of the second control unit 213.

6. The functional health band of claim 4, wherein the remote controller 210 further comprises:
a temperature detection unit 216 configured to measure body temperature of a user,
a pulse detection unit 217 configured to detect a number of pulses,
a body fat measurement unit 218 configured to measure body fat of a user,
an MP3 driving unit 219 configured to provide a music or language study function,
a step counter sensor unit 231 configured to count and store a number of steps when detecting information on a step of a user,
a voice output unit 232 configured to have an earphone 300 for outputting music or language study in a voice form connected thereto, and
a timer unit 234 configured to display the time.

7. The functional health band of claim 1, wherein:
a first pocket 118 is provided on an upper side at the center of the body 112 of the health band unit 110, and
a second pocket 119 into which the main body 120 is inserted is further provided under the first pocket 118.

8. The functional health band of claim 1, wherein permanent magnets 119b are integrally provided in a rear side 119a of the second pocket 119.
